(19) United States
(12) Patent Application Publication
(10) Pub. No.: US 2019/0099344 A1
(43) Pub. Date: Apr. 4, 2019

(54) TOPICAL SPRAY FORMULATION OF GLYCOPYRROLATE

(71) Applicant: Suven Life Sciences Limited, Hyderabad (IN)

(72) Inventors: Mohan Rao Manam, b b (IN); Murali Krishna Prasad Vallabhaneni, b b (IN); Venkateswarlu Jasti, b b (IN)

(21) Appl. No.: 16/091,780

(22) PCT Filed: Apr. 11, 2017
(86) PCT No.: PCT/IB2017/052078

§ 371 Date: Oct. 5, 2018

(30) Foreign Application Priority Data
Apr. 11, 2016 (IN) 201641012622

(51) Int. Cl. A61K 8/49; A61Q 15/00; A61K 8/81; A61K 8/86; A61K 8/34; A61K 8/35; A61K 8/73

(52) U.S. Cl. CPC A61K 8/4913; A61Q 15/00; A61K 8/8176; A61K 8/86; A61K 2800/87; A61K 8/8152; A61K 8/8182; A61K 8/35; A61K 8/731; A61K 8/345

(57) ABSTRACT

The present invention relates to topical spray compositions and methods for the preparation of the same. The topical spray composition comprising Glycopyrrolate or its salts with specific class of excipients may be used to reduce various secretions in human body, specifically to control hyperhydrosis.

### FIELD OF INVENTION

The present invention relates to the field of topical pharmaceuticals. The invention specifically relates to topical spray compositions and methods for the preparation of the same.

The present invention more specifically relates to topical spray compositions of Glycopyrrolate or its salts which enables its uniform application and easy spreading over the body surface. The composition may be used for reducing various secretions in human body, specifically to control hyperhydrosis.

The present invention also relates to the use of the topical compositions of the present invention along with a device.

### BACKGROUND OF INVENTION

Glycopyrrolate is a quaternary amine, which acts as an anti-muscarinic and anti-cholinergic agent administered by oral, parenteral or topical route to reduce various secretions in human body.

The active ingredient of the present invention is Glycopyrrolate bromide chemically known as “3-[(cyclopentylhydroxyphenylacetyl)oxy]-1,1-dimethylpyrrolidinium bromide” and has an empirical chemical formula C₁₉H₂₈BrNO₃ with a structural formula as follows:

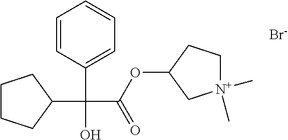 [SMILES: C[N+]1(C)CCC(OC(=O)C(O)(c2ccccc2)C2CCCC2)C1 ; [Br-]]

Glycopyrrolate is an anticholinergic and antispasmodic agent that inhibits gastrointestinal nerve receptor sites that stimulate both the secretion of stomach acid and smooth muscle activity in the digestive tract. Accordingly, it diminishes the volume and free acidity of gastric secretions and controls excessive pharyngeal, tracheal, and bronchial secretions.

U.S. Pat. No. 2,956,062 discloses Glycopyrrolate product as esters of amino alcohols. The invention relates to esters of amino alcohols, more particularly certain esters of N- or 1-substituted 3-pyrrolidinols. The invention also relates to process for the preparation of Glycopyrrolate product.

Glycopyrrolate is reported to be used as effective initial method of treating hyperhidrosis/reducing Sweat and salivary gland secretions. (Refer The American Journal of Digestive Diseases, 1967, 12(5), 439-448; Diabetology, 1997, 40, 299-301; Korean J Pain. 2012 January; 25(1): 28-32).

U.S. Pat. No. 4,534,958 discloses a sprayable aerosol foam treatment composition which is a liquid in the aerosol container and forms a gel upon application to the skin, which comprises water, propellant, volatile solvent and a polyoxyethylene-polyoxypropylene copolymer.

IN 201134 discloses a topical, medicinal spray composition comprising of a drug or combination of drugs as solution or suspension in a volatile carrier containing a polymer or combination of polymer or combination of polymers which when sprayed on the surface of the skin forms a film on skin. It also discloses a topical spray composition which can be sprayed on to the skin to form a breathable film or patch, which films remains stable and in place over a period of days. In this way, medicament can be delivered transdermally over a period of time. However, this patent is silent about the use of glycopyrrolate as a medicament.

U.S. Pat. No. 7,060,289 discloses a convenient and safe product and method of applying glycopyrrolate topically in order to reduce excessive sweating in localized areas for those who suffer from this condition. This invention also discloses combining of oral and topical delivery of glycopyrrolate to reduce excessive sweating and minimize side effects. This also discloses a topical pad containing an amount of glycopyrrolate in solution, for topical application of a therapeutically effective amount of glycopyrrolate, which is useful in reducing sweating in humans.

The present invention attempts to provide Glycopyrrolate specific formulation composition and its spray form enable its uniform application and easy spreading over the body surface. Glycopyrrolate in a carrier which, when sprayed on a surface, forms a film. The applied doses in the form of spray will be dried up and exist as a thin film for a period of time, which does not interfere with perspiration, respiration and other metabolic activities of the skin, so as to therapeutically effective on said part of the human body. The present invention also relates to the use of the topical composition of the present invention along with a device.

### OBJECTIVE OF INVENTION

The objective of the present invention is to formulate and deliver Glycopyrrolate or its salts in the form of topical spray for the treatment of hyperhidrosis and reducing the excessive sweating in localized parts of body.

Another objective of the present invention is to provide the specific formulation composition and its spray form which enables its uniform application and easy spreading over the body surface.

Another objective of the present invention is use of the topical composition of the present invention along with a device.

### SUMMARY OF INVENTION

The present invention provides a topical spray composition of Glycopyrrolate or its salts and methods for the preparation of the same.

The present invention provides a topical spray composition of Glycopyrrolate or its salts for the treatment of hyperhidrosis.

The present invention further relates to a topical spray composition comprising glycopyrrolate or its pharmaceutically acceptable salts thereof and one or more pharmaceutically acceptable excipients.

The present invention further relates to a method of treating hyperhydrosis using a topical spray composition comprising glycopyrrolate or its pharmaceutically acceptable salts thereof and one or more pharmaceutically acceptable excipients.

In one embodiment of the present invention, glycopyrrolate according to the present invention includes but not limited to glycopyrrolate and its pharmaceutically acceptable salts, ethers, esters, polymorphs, prodrugs, derivatives thereof, preferably glycopyrrolate bromide.

Another embodiment of the present invention provides a topical spray composition comprising:

0.1 to 10% w/v of glycopyrrolate,

0.1 to 10% w/v of film formers,

0.1 to 10% w/v of solubilizers/surfactants,

0.1 to 5.0% w/v of permeation enhancers,

0.1 to 10% w/v of plasticizers,

0.1% to 10% of other additives and

a vehicle q.s to 100%.

Another embodiment of the present invention provides a topical spray composition comprising:

0.1 to 10% w/v of glycopyrrolate,

0.1 to 10% w/v of film formers,

0.1 to 10% w/v of solubilizers/surfactants,

0.1 to 5.0% w/v of permeation enhancers,

0.1 to 10% w/v of plasticizers,

0.1% to 10% of other additives,

optionally containing a propellant and

a vehicle q.s to 100%.

Another embodiment of the present invention provides a topical spray composition comprising:

0.1 to 10% w/v of glycopyrrolate bromide,

0.1 to 10% w/v of film formers,

0.1 to 10% w/v of solubilizers/surfactants,

0.1 to 5.0% w/v of permeation enhancers,

0.1 to 10% w/v of plasticizers,

0.1% to 10% of other additives and

a vehicle q.s to 100%.

Another embodiment of the present invention provides a topical spray composition comprising:

0.1 to 10% w/v of glycopyrrolate bromide,

0.1 to 10% w/v of film formers,

0.1 to 10% w/v of solubilizers/surfactants,

0.1 to 5.0% w/v of permeation enhancers,

0.1 to 10% w/v of plasticizers,

0.1% to 10% of other additives,

optionally containing a propellant and

a vehicle q.s to 100%.

Another embodiment of the present invention provides a process for preparing glycopyrrolate topical spray composition, the process comprising steps of:

a. dissolving the film formers in the chosen vehicle with stirring to form a clear solution,

b. dissolving or suspending the drug and one or more pharmaceutically acceptable excipients in the solution of step (a),

c. adding the plasticizer to the solution of step (b) and filling the mixture in a conventional aerosol can, and

d. charging the filled can with liquefied propellant.

Another embodiment of the present invention provides a process for preparing glycopyrrolate topical spray composition, the process comprising steps of:

a. dissolving the film formers in the chosen vehicle with stirring to form a clear solution,

b. dissolving or suspending the drug and one or more pharmaceutically acceptable excipients in the solution of step (a), and

c. adding the plasticizer to the solution of step (b) and filling the mixture in a metered dose pump.

Yet another embodiment of the present invention provides a topical spray composition of Glycopyrrolate or its salts and methods for the preparation of the same which optionally contains propellant for treatment of hyperhidrosis.

Yet another embodiment of the present invention provides a specific formulation composition and its spray form of Glycopyrrolate or its salts which enable its uniform application and easy spreading over the body surface.

### DETAILED DESCRIPTION OF THE INVENTION

The present invention provides a specific formulation composition and its spray form which enable its uniform application and easy spreading over the body surface. Glycopyrrolate or its salts in a carrier which, when sprayed on a surface, forms a film. The applied doses in the form of spray will be dried up and exist as a thin film for a period of time, which does not interfere with perspiration, respiration and other metabolic activities of the skin, so as to therapeutically effective on said part of the human body. The areas to spray which may includes armpits, face, hands, forehead, neck, scalp, feet, groin, trunk and back of the knees. This method of application is more convenient and safe to use topically with minimized side effects. The topical films formed by this present proposal may show excellent stability, peelability and easy to remove from the site of application.

The spray compositions are preferably in a form suitable for application by spraying from an aerosol or pump spray container. Preferably, the composition is dispensed from a pump dispenser or from an aerosol dispenser. In the latter case, the composition additionally comprises from about 10% to 90% of propellant in order to provide a suitable pressure within the aerosol dispenser. Generally, propellant is not required for compositions dispensed from a pump dispenser. However, if desired, such compositions may also comprise from about 10% to 90% of a propellant which is liquid at room temperature, for example, trichloro-monofluoromethane. The proposed composition contains glycopyrrolate drug along with additional components like film former (a polymer or combination of polymers), solubilizers, enhancers, plasticizers, humectants and fragrances. The drug in the composition may be present in a form that is solubilized or suspended.

The compositions are generally prepared by mixing the ingredients, without liquefied propellant. If propellant is to be added, the resulting mixture is then charged with the liquefied propellant into an aerosol dispenser to achieve the final composition. Dissolve the film formers in the chosen vehicle with stirring to form a clear solution; dissolve or suspend the drug and other ingredient(s) like solubilizers, enhancers, humectants and their combinations. Add the plasticizer to the solution and fill a conventional aerosol can with the mixture; and Charge the filled can with liquefied propellant. Alternatively, the mixed composition is placed in a pump dispenser, for example, a metered dose pump, which dispenses the composition typically without liquefied propellant since a pressurized atmosphere is not required. Propellant which is liquid at room temperature may, however, be included in a pump dispenser composition as part of the aqueous vehicle. Necessary fragrances can be added to impart fragrance to the formulation.

Pharmaceutically acceptable counter salts may be prepared from inorganic and organic acids. Salts derived from inorganic acids include hydrochloric acid, hydrobromic acid, hydrogen fluoride, hydrogen iodide, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid (tosylate), salicylic acid, and the like.

Glycopyrrolate can be delivered more easily and effectively to control the excess sweating. This invention includes:

i) Topical formulation comprising Glycopyrrolate as a solution or suspension in a vehicle optionally containing a polymer or combination of polymers which, when sprayed on the surface of the skin, forms a film on the skin.

ii) The composition may further contain one or more solubilizer, permeation enhancers, plasticizers and humectants.

iii) The composition may further contain one or more fragrances.

iv) Further the compositions can be dispensed from any dispenser, preferably a dispenser which provides the composition as a spray and the drug from the composition may be released over a period of time.

v) Preferably, the composition is dispensed from a pump dispenser or from an aerosol dispenser.

vi) When the composition is dispensed as an aerosol, the vehicle partly comprises a propellant to built necessary pressure, which may includes dichlorotetrafluoroethane (P114), difluoroethane (P152a), tetrafluoroethane (134a), heptafluoropropane (P227b), trichloromonofluoromethane (P11), dichlorodifluoromethane (P12), monochlorodifluoromethane (P22); or compressed gases.

vii) The present spray formulations of Glycopyrrolate can be applied for the purpose of improving the hygienic in all human beings.

viii) This can be used to control/reduce the sweating in the areas to spray which may includes armpits, groin, face, neck, hands, forehead, scalp, feet, trunk and back of the knees.

The composition of the present invention is administered using any conventional device which is used for topical sprays for forming a film.

The aerosol dispenser is preferably a conventional aerosol can having a conventional metered spray aerosol valve. The pump dispenser is preferably a conventional can or bottle having a conventional metered spray pump. Preferably, the aerosol dispenser has an all position valve having a shroud that permits spraying when the dispenser is held at any angle. In this way, horizontal bottom surfaces as well as horizontal top surfaces and vertical surfaces can be sprayed. The valve actuator can be any actuator which produces a spray and not a foam at the nozzle. A preferred valve actuator is a mechanical breakup actuator, which employs mechanical forces rather than expansion and evaporation of the propellant to produce a spray. A typical mechanical breakup actuator has a conical or cylindrical swirl chamber with an inlet channel oriented perpendicular to the axis thereof. This structure imparts a swirling motion to the aerosol mixture upon discharge. The swirling motion occurs around the axis of the swirl chamber forming a then conical film of discharged mixture, which breaks into droplets as it leaves the swirl chamber and travels in the direction of the axis thereof. The result is a fine, soft, dispersed spray which can be easily controlled to produce a stable thin film of even thickness completely contacting the application site. In dispensing a composition of the invention the dispenser is typically held about 1 to 2 inches (2.5 to 5 cm) from the application site and produces a film of even thickness. The dispensers used in the present invention are preferably compact units.

The film-formers referred above preferably include any acrylic polymers or copolymers. Preferred film-formers include a non-ionic copolymer of methyl methacrylate and butyl methacrylate (Plastoid B®), a copolymer of dimethylamine ethyl methacrylate and a neutral methacrylic acid ester (Eudragit E 100®), amino methacrylate copolymer type B (Eudragit RS®, USP/NF), amino methacrylate copolymer type A (Eudragit RL®, USP/NF), methacrylic acid copolymer type A (Eudragit L100®, USP/NF), methacrylic acid copolymer type B (Eudragit S100®, USP/NF), polyvinyl acetate, cellulose acetate, polyvinyl alcohol, povidone, povidone vinyl acetate, hydroxypropyl methyl cellulose, Chitins, Glucans, hydroxy ethyl cellulose, methyl cellulose, Carboxy methyl cellulose, Hydroxy propyl cellulose, Carageenan and Chitosan or combinations thereof.

Preferred solubilizers referred above include a copolymer of dimethylamine ethyl methacrylate and a neutral methacryclic acid ester (Eudragit E 100®, USP/NF); a non-ionic copolymer of methyl methacrylate and butyl methacrylate; surfactants, for example , sodium lauryl sulphate; polyhydric alcohols, for example, propylene glycol or polyethylene glycols: vitamin E, vitamin E TPGS (tocopheryl polyethylene glycol 1000 succinate), labrasol, or any two or more of the above in combination, Preferably, the solubiliser is a copolymer of dimethylamine ethyl methacrylate and a neutral methacrylic acid ester (Eudragit E100®) in combination with, a non-ionic copolymer of methyl methacrylate and butyl methacrylate (Plastoid B®). The solubilizers serve to dissolve the drug in the chosen vehicle. Many of the solubilizers also enhance percutaneous penetration of drug and/or act as humectants.

Preferred plasticisers referred above include triethyl citrate, dimethyl isosorbide, acetyltributyl citrate, castor oil, propylene glycol, polyethylene glycol and combination thereof.

The permeation enhancer referred above is preferably a lyophilic solvent, for example, dimethyl sulfoxide, dimethyl formamide or isopropyl myristate; a surfactant, for example. Tween 80 or sodium lauryl sulfate or menthol; a two-component system, for example, oleic acid and octyl dimethyl paraamino benzoic acid (Padimate 0); or a polyhydric alcohol for example, propylene glycol or diethylene glycol monoethyl ether EP (transcutol): or any two or more of the above in combination.

The vehicle referred above can be water or a non-aqueous solvent. Preferred nonaqueous vehicles include acetone, isopropyl alcohol, methylene chloride , methyl-ethyl-ketone, absolute alcohol, ethyl acetate, 2-(2-Ethoxyethoxy)ethanol and trichloromonofluromethane (P11): or any two or more of the above in combination.

The aqueous or non-aqueous vehicle may additionally—comprise (weight/weight of vehicle) from about 1% to 20% of one or more humectants. Preferred humectants include polyhydric alcohols and polyvinylprrolidone. Preferred polyhydric alcohols are propylene glycol, butylenes glycol, polyethylene glycols, glycerol and sorbitol.

The water-soluble additive referred above is preferably propylene glycol, sodium lauryl sulphate, one or more polaxomers, polyoxyl 34 castor oil, polyoxyl 40 hydrogenated castor oil, cetomacrogol, polyethylene glycol or transcutol; or any two or more of the above in combination.

When the composition is dispensed as an aerosol, the vehicle partly comprises a propellant in an amount to provide from about 10% to 90% (w/v) of the composition.

The propellant can be any pharmaceutically acceptable propellant which provides a pressure of from about 20 p.s.i.g. to about 130 p.s.i.g. within an aerosol dispenser. Preferred propellants include hydrocarbons, for example, propane, butane, isobutene, or dimethylether; hydrofluorocarbons and hydrochlorofluorocarbons, for example dichlorodifluoromethane (P12), trichloromonofluoromethane (P11), dichlorofluoroethane, monochlorodifluoromethane (P22), dichlorotetrafluoroethane (P114), difluoroethane (P152a), tetrafluoroethane (P134a), heptafluoropropane (P227b), or compressed gases, for example, nitrogen or carbon dioxide.

The following examples describes the nature of the invention and are given only for the purpose of illustrating the present invention in more detail and are not limitative and relate to solutions which have been particularly effective on a bench scale:

### EXAMPLE 1

| S. No. | Ingredients | % w/v |
|---|---|---|
| 1. | Glycopyrrolate | 0.25-6.0 |
| 2. | Povidone K 30 | 3.0 |
| 3. | PEG 400 | 1.5 |
| 4. | Propylene glycol | 1.0 |
| 5. | Ethanol | 15.0 |
| 6. | 2-(2-Ethoxyethoxy)ethanol | 1.0 |
| 7. | Acetone | 15.0 |
| 8. | Propellant/Propellant mixture | Qs. |
| 9. | Fragrance | Qs. |

### EXAMPLE 2

| S. No. | Ingredients | % w/v |
|---|---|---|
| 1. | Glycopyrrolate | 0.25-6.0 |
| 2. | Dimethylaminoethyl Methacrylate Copolymer | 3.0 |
| 3. | PEG 400 | 1.5 |
| 4. | Propylene glycol | 1.0 |
| 5. | Acetone | 15.0 |
| 6. | Ethanol | 15.0 |
| 7. | 2-(2-Ethoxyethoxy)ethanol | 1.0 |
| 8. | Propellant/Propellant mixture | Qs. |
| 9. | Fragrance | Qs. |

### EXAMPLE 3

| S. No. | Ingredients | % w/v |
|---|---|---|
| 1. | Glycopyrrolate | 0.25-6.0 |
| 2. | Polyvinylpyrrolidone vinyl acetate | 3.0 |
| 3. | Propylene glycol | 1.0 |
| 4. | PEG 400 | 1.5 |
| 5. | Isopropyl alcohol | 15.0 |
| 6. | 2-(2-Ethoxyethoxy)ethanol | 1.0 |
| 7. | Propellant/Propellant mixture | Qs. |
| 8. | Fragrance | Qs. |

### EXAMPLE 4

| S. No. | Ingredients | % w/v |
|---|---|---|
| 1. | Glycopyrrolate | 0.25-6.0 |
| 2. | Polyvinylpyrrolidone | 2.0 |
| 3. | Polyvinylpyrrolidone vinyl acetate | 1.0 |
| 4. | PEG 400 | 1.5 |
| 5. | 2-(2-Ethoxyethoxy)ethanol | 1.0 |
| 6. | Acetone | 15.0 |
| 7. | Ethanol | 15.0 |
| 8. | Propellant/Propellant mixture | Qs. |
| 9. | Fragrance | Qs. |

### EXAMPLE 5

| S. No. | Ingredients | % w/v |
|---|---|---|
| 1. | Glycopyrrolate | 0.25-6.0 |
| 2. | Carboxy methyl cellulose | 3.0 |
| 3. | Propylene glycol | 1.0 |
| 4. | PEG 400 | 1.5 |
| 5. | 2-(2-Ethoxyethoxy)ethanol | 1.0 |
| 6. | Acetone | 20 |
| 7. | Isopropyl alcohol | Q.s. |
| 8. | Fragrance | Qs. |

### EXAMPLE 6

| S. No. | Ingredients | % w/v |
|---|---|---|
| 1. | Glycopyrrolate | 0.25-6.0 |
| 2. | Polyvinylpyrrolidone/vinyl acetate | 3.0 |
| 3. | Propylene glycol | 1.0 |
| 4. | PEG 400 | 1.5 |
| 5. | 2-(2-Ethoxyethoxy)ethanol | 1.0 |
| 6. | Ethanol | 25.0 |
| 7. | Methylene chloride | 20.0 |
| 8. | Acetone | Q.s. |
| 9. | Fragrance | Qs. |

### EXAMPLE 7

| S. No. | Ingredients | % w/v |
|---|---|---|
| 1. | Glycopyrrolate | 0.25-6.0 |
| 2. | Carboxy methyl cellulose | 3.0 |
| 3. | PEG 400 | 1.5 |
| 4. | Propylene glycol | 1.0 |
| 5. | 2-(2-Ethoxyethoxy)ethanol | 1.0 |
| 6. | Ethanol | 25.0 |
| 7. | Methylene chloride | 20.0 |
| 8. | Acetone | Q.s. |
| 9. | Fragrance | Q.s. |

### EXAMPLE 8

| S. No. | Ingredients | % w/v |
|---|---|---|
| 1. | Glycopyrrolate | 0.25-6.0 |
| 2. | Carageenan | 3.0 |
| 3. | PEG 400 | 1.5 |
| 4. | Propylene glycol | 1.0 |
| 5. | 2-(2-Ethoxyethoxy)ethanol | 1.0 |
| 6. | Ethanol | 25.0 |
| 7. | Methylene chloride | 20.0 |
| 8. | Acetone | Q.s. |
| 9. | Fragrance | Qs. |

### EXAMPLE 9

| S. No. | Ingredients | % w/v |
|---|---|---|
| 1. | Glycopyrrolate | 0.25-6.0 |
| 2. | Hydroxy propyl cellulose | 3.0 |
| 3. | PEG 400 | 1.5 |
| 4. | Propylene glycol | 1.0 |
| 5. | 2-(2-Ethoxyethoxy)ethanol | 1.0 |
| 6. | Ethanol | 25.0 |
| 7. | Methylene chloride | 20.0 |
| 8. | Acetone | Q.s. |
| 9. | Fragrance | Qs. |

### EXAMPLE 10

| S. No. | Ingredients | % w/v |
|---|---|---|
| 1. | Glycopyrrolate | 0.25-6.0 |
| 2. | Chitosan | 3.0 |
| 3. | PEG 400 | 1.5 |
| 4. | Propylene glycol | 1.0 |
| 5. | 2-(2-Ethoxyethoxy)ethanol | 1.0 |
| 6. | Ethanol | 25.0 |
| 7. | Methylene chloride | 20.0 |
| 8. | Acetone | Q.s. |
| 9. | Fragrance | Qs. |

1. A topical spray composition comprising:
0.1 to 10% w/v of glycopyrrolate or its pharmaceutically acceptable salts,
0.1 to 10% w/v of film formers,
0.1 to 10% w/v of solubilizers/surfactants,
0.1 to 5.0% w/v of permeation enhancers,
0.1 to 10% w/v of plasticizers,
0.1% to 10% of other additives,
optionally containing 10 to 90% v/v of a propellant, and
a vehicle q.s to 100%.

2. The composition as claimed in claim 1, wherein said film-former is selected from the group consisting of a non-ionic copolymer of methyl methacrylate and butyl methacrylate, a copolymer of dimethylamine ethyl methacrylate and a neutral methacrylic acid ester, amino methacrylate copolymer type B, amino methacrylate copolymer type A, methacrylic acid copolymer type A, methacrylic acid copolymer type B, polyvinyl acetate, cellulose acetate, polyvinyl alcohol, povidone, povidone vinyl acetate, hydroxypropyl methyl cellulose, Chitins, Glucans, hydroxy ethyl cellulose, methyl cellulose, Carboxy methyl cellulose, Hydroxy propyl cellulose, Carageenan and Chitosan and combinations thereof.

3. The composition as claimed in claim 1, wherein said solubilizer/surfactant is selected from the group consisting of a copolymer of dimethylamine ethyl methacrylate and a neutral methacryclic acid ester, a non-ionic copolymer of methyl methacrylate and butyl methacrylate sodium lauryl sulphate; propylene glycol, polyethylene glycols, vitamin E, vitamin E TPGS (tocopheryl polyethylene glycol 1000 succinate), labrasol, and combinations thereof.

4. The composition as claimed in claim 1, wherein said plasticiser is selected from the group consisting of triethyl citrate, dimethyl isosorbide, acetyltributyl citrate, castor oil, propylene glycol, polyethylene glycol and combination thereof.

5. The composition as claimed in claim 1, wherein said permeation enhancer is selected from the group consisting of dimethyl sulfoxide, dimethyl formamide, isopropyl myristate, Tween 80, sodium lauryl sulfate, menthol, a mixture of oleic acid and octyl dimethyl paraamino benzoic acid, propylene glycol, diethylene glycol monoethyl ether and combination thereof.

6. The composition as claimed in claim 1, wherein said vehicle is selected from the group consisting of water, acetone, isopropyl alcohol, methylene chloride, methyl-ethyl-ketone, absolute alcohol, ethyl acetate, 2-(2-Ethoxyethoxy)ethanol, trichloromonofluromethane and combination thereof.

7. The composition as claimed in claim 1, wherein said water-soluble additive is selected from the group consisting of propylene glycol, sodium lauryl sulphate, polaxomers, polyoxyl 34 castor oil, polyoxyl 40 hydrogenated castor oil, cetomacrogol, polyethylene glycol, transcutol and combination thereof.

8. The composition as claimed in claim 1, wherein said propellant is selected from dichlorotetrafluoroethane (P114), difluoroethane (P152a), tetrafluoroethane (134a), heptafluoropropane (P227b), trichloromonofluoromethane (P11), dichlorodifluoromethane (P12), monochlorodifluoromethane (P22),compressed gases and combinations thereof.

9. A process for preparing glycopyrrolate topical spray composition as claimed in claim 1, the process comprising steps of:
a. dissolving the film formers in the chosen vehicle with stirring to form a clear solution,
b. dissolving or suspending the drug and one or more pharmaceutically acceptable excipients in the solution of step (a),
c. adding the plasticizer to the solution of step (b) and filling the mixture in a conventional can, and
d. charging the filled can with liquefied propellant or filling the mixture in a metered dose pump.

10. A process for preparing glycopyrrolate topical spray composition as claimed in claim 1, the process comprising steps of:
a. dissolving the film formers in the chosen vehicle with stirring to form a clear solution,
b. dissolving or suspending the drug and one or more pharmaceutically acceptable excipients in the solution of step (a), and
c. adding the plasticizer to the solution of step (b) and filling the mixture in a metered dose pump.

11. The use of topical spray composition as claimed in claim 1 comprising glycopyrrolate or its pharmaceutically acceptable salts thereof and one or more pharmaceutically acceptable excipients for treating hyperhydrosis.

12. A topical spray composition as claimed in claim 1, wherein the glycopyrrolate or its pharmaceutically acceptable salts comprises glycopyrrolate bromide.

13. A topical spray composition as claimed in claim 1, further comprising one or more pharmaceutically acceptable excipients.
